(19) United States
(12) Patent Application Publication
(10) Pub. No.: US 2025/0204931 A1
(43) Pub. Date: Jun. 26, 2025

(54) FLEXIBLE SURGICAL TOOL WITH INTEGRATED BEARING ASSEMBLY

(71) Applicant: Aesculap AG, Tuttlingen (DE)

(72) Inventors: Andre Buerk, Villingen-Schwenningen (DE); Thomas Hagen, Tuttlingen (DE); Simone Hermle, Villingen-Schwenningen (DE); Lilian Killinger, Ehningen (DE); Juergen Barth, Denkingen (DE); Roland-Alois Hoegerle, Tuttlingen (DE); Uwe Schaz, Neuhausen (DE)

(21) Appl. No.: 18/851,753

(22) PCT Filed: Mar. 28, 2023
(86) PCT No.: PCT/EP2023/058017

§ 371 Date: Sep. 27, 2024

(30) Foreign Application Priority Data
Apr. 4, 2022 (DE) 10 2022 107 970.8

(51) Int. Cl. A61B 17/16

(52) U.S. Cl. CPC A61B 17/162; A61B 17/1631; A61B 17/1671

(57) ABSTRACT

A surgical tool is adapted to be inserted into an instrument shaft of a surgical instrument. The tool includes a distal tool tip, a tool shaft extending from the distal tool tip in a proximal direction and having a distal bearing portion as well as a drive portion arranged proximally to the distal bearing portion. The drive portion is formed for positive engagement with a drive shaft of the surgical instrument. A bearing bush is mounted on the distal bearing portion and has a bearing coupling element for releasable coupling with an instrument shaft of the surgical instrument.

### CROSS-REFERENCE TO RELATED APPLICATION(S)

This application is the United States national stage entry of International Application No. PCT/EP2023/058017, filed on Mar. 28, 2023, and claims priority to German Application No. 10 2022 107 970.8, filed on Apr. 4, 2022. The contents of International Application No. PCT/EP2023/058017 and German Application No. 10 2022 107 970.8 are incorporated by reference herein in their entireties.

### FIELD

The present disclosure relates to a surgical instrument and a surgical tool mounted or mountable therein.

### BACKGROUND

In surgical technique, it is advantageous to angle the distal shaft portion of a shaft of a handheld medical instrument. This allows surgical operations to be performed in small space, for example during surgeries on the spinal column.

Instruments that allow the distal tip to be angled have been known for some time in the field of surgical robots. This enables precise movement of the instruments in the tightest of spaces. However, no rotating tools are angled with these instruments. One example of this is the “Da Vinci” surgical robot from Intuitive Surgical.

There are various angled medical devices available on the market. For example, Human Xtensions has developed angled forceps. A surgeon can use hand-held robotic instruments to translate “rough” hand movements into delicate movements at the tip of the instrument. With this instrument, an instrument tip can be angled over a flexible portion that extends over a length of approx. 20 mm. The flexible portion is supported by a kind of plastic stent. Adjustment is made by means of wire strands that are guided past the outside of the stent. The disadvantage here is the length of the bending area and the very flexible tip. The flexibility is caused by the plastic stent and the wire strands.

Furthermore, there are already manufacturers of cutting handpieces/medical hand instruments, which allow to be angled. These are primarily made use of in endoscopic procedures on the spine and enable minimally invasive techniques as well as simple processing of structures that are difficult to access in this area. The joint constructions of these cutting handpieces have a rather open design and a certain degree of angular flexibility. This means that the angle changes slightly when pressure is applied to the tip of the cutter. Furthermore, only angles of up to 36° are possible with these cutting handpieces. An example of this are cutting handpieces marketed under the registered trademark JOIMAX®.

Moreover, there are high-speed cutting handpieces/medical hand instruments with angled shafts. Thanks to interchangeable shafts, a handpiece can be selected from three variants: 0°, 7.5° and 15° of angulation. The biggest disadvantage is the large bending radius through which angulation is realized. This takes up a lot of space and limits the options for action in the surgical field. In addition, the angle cannot be adjusted intraoperatively. Due to the fixed angulation, endoscopic work with angled shafts is not possible, as they cannot be inserted into the straight working channel of the endoscope. Furthermore, a maximum angulation of 15° is not particularly large.

From disclosures DE 10 2017 010 033 A1 and U.S. Pat. No. 10,178,998 B2 cutters with heads that can be angled are known. Both solutions are implemented through fork joints.

### SUMMARY

The present disclosure is based on the task of reducing or avoiding disadvantages of the prior art. In particular, a surgical tool and/or a surgical instrument and/or an associated system is/are to be provided, which is simple and inexpensive and enables precise guidance at high speeds.

The problem underlying the invention is solved by a surgical tool, a surgical instrument and an associated system. Advantageous embodiments will be described in more detail later.

More specifically the problem is solved by a surgical tool that is adapted to be inserted into the instrument shaft of a surgical instrument. The tool has a distal tool tip, such as a drill or cutter. Furthermore, the tool has a tool shaft that extends from the distal tool tip in a proximal direction and has a distal bearing portion and a drive portion arranged proximally thereto. The drive portion is adapted for positive engagement with a drive shaft of the surgical instrument. Moreover, the tool has a bearing bushing mounted on the distal bearing portion and a bearing coupling element for releasable coupling with an instrument shaft, in particular a distal instrument shaft portion, of the surgical instrument.

In other words, a surgical tool for integration into a surgical instrument, in particular a handpiece/hand instrument, is provided including an integrated bearing assembly. The tool has a tool head or a tool tip adapted, for example, to work on human tissue, bone, etc., and a tool shaft at the distal end of which the tool head/tool tip is located. The tool shaft is adapted to be inserted (from a distal direction) into an instrument shaft of a surgical instrument, in particular a hand instrument, and to be coupled therein with a drive provided by the instrument for driving the tool tip/tool head. Furthermore, the tool has a bearing element/bearing bush attached (in particular directly) proximally from the tool head/tool tip to the tool shaft, which can be fixed together with the tool on/in the instrument shaft.

More specifically, the surgical tool, in particular the tool shaft and the bearing bush, is adapted to be inserted into the distal end of an instrument shaft. In particular, in a state in which the surgical tool is not inserted into the surgical instrument the bearing bushing is mounted to the bearing portion of the tool shaft in an axially fixed manner. In other words, in a state in which the tool is present individually, i.e. in which it is not inserted into the instrument shaft, the bearing bush preferably is at least connected to the bearing portion of the tool shaft in an axially fixed manner. Preferably, the bearing bush has a larger diameter than the entire tool shaft proximal to the bearing bush, preferably than an entire section of the tool proximal to the bearing bush. In particular, the bearing bush has a larger diameter than the drive portion of the tool shaft. Further preferably, the entire bearing bush is arranged distally from the drive portion. Preferably, the bearing bush has an inner ring, which is connected to the bearing portion in a substantially axially and rotationally fixed manner. Further preferably, the bearing bush has an outer ring that is provided in a rotatable manner (and axially fixed apart from a bearing clearance) to the inner ring. The bearing coupling element is preferably provided on the outer ring, in particular on a radial outer surface of the outer ring. Preferably, the tool has a seal at a distal end of the bearing bush or distally from the bearing bush, in particular in the form of a cover disk. This can reduce or prevent in particular that dirt and/or fluid penetrate into the bearing bush and into the instrument shaft. Preferably, only the tool tip and possibly a seal are positioned distally from the bearing bush. Preferably, apart from possibly a seal, no components are arranged distally from the bearing bush which interact directly with the instrument shaft located radially outside thereof.

In such tools and instruments, a high speed at a low temperature at the same time and precise positioning of the tool tip/head is made possible, which always has a constant projection over a distal end of the instrument shaft. The special tool design enables precise tool guidance, undisturbed running and optimized tool coupling in the instrument housing. Furthermore, the tool is particularly easy to assemble and disassemble.

Preferably, the tool shaft also has a flexurally flexible and torsion-resistant flex portion, which is arranged between the bearing portion and the drive portion. This enables the tool tip to be angled in relation to the tool shaft and the instrument shaft while at the same time ensuring precise positioning of the tool and very smooth running. The flexible part or flex portion ensures that the tool can be operated both in a 0° position and in an angled position (e.g.) 45° and can transmit torque. For example, the flex portion can be connected to the bearing portion at its distal end, e.g. via a fork or mount of the bearing section. Alternatively or additionally, the flex section can be connected to the drive portion at its proximal end, for example via a fork or mount of the drive portion. A section of the bearing portion that is thickened in the region of the fork or mount at the same time may serve as a radial shoulder for contact of the bearing bush.

In particular, an angulation of the tool tip with respect to the tool shaft of 30-60°, preferably at least 45°, is provided. The fact that the curvature of the flex portion is determined from the outside by an angulation of the tool shaft means that only a small axial length is required for the angulation.

Such a flex portion can be provided, for example, by a special braiding of individual strands. The flex portion can have a torque strand, in particular with several layers of alternating winding direction. In other words, the flex portion can have a first sleeve made of individual strands or individual fibers wound in a first direction and a second sleeve (firmly connected to it) made of individual strands or individual fibers wound in a second direction. In this way, clockwise and anticlockwise rotation can be realized without the strand untwisting and destroying itself.

In particular, the drive portion and/or the bearing portion are adapted to be bend-proof and torsion-resistant, for example in the form of a rod or tube. This enables a particularly precise coupling to the drive of the instrument or a particularly good support via the bearing bush.

Further in particular, the surgical tool is a tool to be driven in rotation, especially a cutting or drilling tool.

It is advantageous if the bearing bush has a proximal front side, which forms a stop surface for contact in the instrument shaft, in particular in its distal shaft portion. This makes it possible to precisely define a position of the tool tip relative to the instrument shaft so that a constant projection of the tool tip over a distal end of the instrument shaft is ensured for all adjustable angles of the tool tip relative to the tool shaft. In addition, a stop surface provided proximally on the bearing bush prevents a corresponding load from being transferred to the flex portion when the tool tip is loaded and the flex section from being compressed or too heavily loaded during operation.

Preferably, the bearing coupling element forms a radial recess or a radial projection on or in a circumferential wall of the bearing bush. For example, a radial recess can be provided in the form of a circumferential groove. A suitable counter-coupling element or a latching mechanism (for example with a latching body in form of a ball pressure element) can be provided radially inside on the instrument shaft in order to engage or latch with the radial recess or the radial projection, for example in the style of a click connection. This enables particularly quick and easy assembly and, in particular, simple positioning of the bearing bush on the instrument shaft in the axial direction. The housing of the bearing bush ensures safe and easy insertion of the tool into the shaft during the coupling process.

Further preferably, the bearing bush provides a single-row, even more preferably a double-row, roller bearing including one or two roller bearing rings (a miniature roller bearing/ball bearing). The roller bearing can have deep groove ball bearings or angular ball bearings, the latter having the advantages of better load absorption and easier assembly. This makes them particularly suitable for high speeds and low tool temperatures at the same time. In the case of a double-row roller bearing, particularly smooth running and a particularly low temperature of the tool are achieved and the risk of the tool tilting can be minimized to a particularly high degree. As a result, the tool is particularly stable and the flex portion is particularly little loaded.

In particular, the bearing bush in a double-row roller bearing has a radially inner spacer bushing and/or a preload element, which determine the axial position of the two roller bearing rings in relation to each other. In other words, the spacer bushing can be arranged between the individual bearings and can be in contact with them, for example on their inner rings, in order to position them relative to each other. The preload element can be, for example, a wave spring washer and can be inserted between one of the bearings and a housing section of the bearing bush. Thus, the preload element can preload the corresponding adjacent bearing outwards in the axial direction in relation to the respective other bearing. In this way, component tolerances can be compensated, the service life of the bearings or the bearing bush can be increased and the noise level can be reduced. Preferably, the bearing bush can have a cover disk, in particular on its distal side, which protects a correspondingly adjacent bearing from mechanical damage and possibly from the penetration of a liquid (in case of an implementation as a sealing washer).

Preferably, the bearing bush is mounted in the axial direction between the bearing portion and the distal tool tip, e.g. between a first circumferential step of the bearing portion and a second circumferential step of the distal tool tip. For example, the distal tool tip can be screwed with the bearing portion. For example, the distal tool tip can form an internal thread, which is screwed onto an external thread of the bearing portion. In this way no additional elements need to be provided in order to fasten the bearing bush to the tool shaft. Optionally, the bearing portion can be (integrally) connected to the tool tip. The bearing bush can be fastened to the bearing portion via a screw nut or the like.

It has proven to be advantageous if the drive portion forms at least one engagement portion extending in the axial direction, in particular a profile shaft portion, such as e.g. a hexagonal profile. A section extending in the axial direction, in particular with a cross-sectional profile that is constant along the axial direction, enables longitudinal displacement between the drive portion and the drive shaft, which can occur, for example, when the flex portion is angled. Moreover, a profile shaft portion is particularly suited for absorbing a drive torque. In the coupled state, the tool can therefore be positively connected to a tube (the drive shaft), which can be driven by a motor in the instrument/handpiece/in an instrument handle. The tube can be firmly connected to the engagement reception, which has a hexagonal geometry, for example, over which the entire tool can be rotated and, for example, a cutting head can remove material.

Preferably, a length of the engagement portion corresponds to at least a maximum displacement distance of a proximal end of the flex portion at its maximum curvature. Thus, at any degree of bending of the tool tip relative to the tool shaft, an overlap and thus a torque transmission between the engagement portion and an engagement reception of the drive shaft with which the engagement portion cooperates can be safeguarded. In other words, a displacement/change of length may occur at the proximal area of the tool. The reason for this is the fixation between the distal end of the instrument shaft and the tool via the bearing bush as well as a change in tool guidance in the angled state and a resulting change in the bending line of the flex portion. A corresponding length compensation at the proximal area is achieved by a length (expansion in axial direction) of the driven engagement portion/profile shaft portion/hexagon. In other words, the engagement portion and the engagement reception are formed such that they can be displaced essentially freely/without stops relative to each other in the axial direction. The resulting reduced driven length of the engagement portion is a few millimeters and can be taken account of in the overall length of the engagement portion in order to ensure reliable transmission of the torque.

Advantageously, the drive portion forms a proximal centering end, in particular a centering tip with beveled side walls, or a beveled circumferential wall. This makes it easy to insert the drive portion into the drive shaft, as the centering end can compensate for misalignments in between. Further preferably, the centering end forms a profile portion, in particular with a three-sided pyramid shape, which is adapted to engage with a counter profile portion of the surgical instrument in a manner determined by the rotational position. A profile portion of the proximal centering end is to be understood as a portion that has a profiling (i.e. a shape that is not continuously rotationally symmetrical) that is at least partially aligned in the axial direction. This can ensure that the profile portion can be associated with a matching counter profile portion only in one (or a certain number of) specific rotational positions.

Alternatively or additionally, the problem underlying the present disclosure is solved by a surgical instrument having an instrument shaft, which is open distally and adapted to receive a surgical tool. The instrument shaft has a distal instrument shaft portion in which a counter coupling element or a latching mechanism is provided, which is designed for releasable coupling with the bearing coupling element of the surgical tool. In particular, the latching mechanism is a spring-biased or preloadable latching mechanism. In other words, it can have an engagement element that is linked to the distal instrument shaft portion via a spring element and can be prestressed or is pretensioned by the spring element, in particular radially inwards. Furthermore, the instrument shaft has a proximal instrument shaft portion having a drive shaft that is adapted for positive engagement with the drive portion of the surgical tool.

In particular, the instrument is a surgical instrument or handpiece with a fully integrated rotary mechanism.

Preferably, the counter coupling element/latching mechanism has a resiliently mounted engagement element/latching body. Such a latching mechanism can latch with the bearing bush in the manner of a click connection and thus provide tactile and/or audible feedback for complete insertion of the tool into the instrument. This means that the tool is ready for use when the axial fixation of the tool has taken place at the end of the coupling process.

Further preferably, the distal instrument shaft portion and the proximal instrument shaft portion can be bent towards each other. In particular, the distal instrument shaft portion and the proximal instrument shaft portion are beveled on mutually facing end faces and can be rotated relative to one another in order to achieve angulation of the instrument shaft. Preferably, a gear train for rotating the distal instrument shaft portion relative to the proximal instrument shaft portion is arranged radially outside, in particular at a distance, from the tool shaft and/or the drive shaft.

It has also proven to be advantageous if the drive shaft forms a distal centering receptacle, in particular in the form of a funnel with beveled side walls. This further improves centering/compensation of misalignments when inserting the tool (the drive portion) into the instrument (into the drive shaft). Preferably, the centering receptacle extends from a hollow shaft first radially outwards and then in a funnel-like inclined manner in a proximal and radially outwards direction. This ensures that the funnel geometry has a radial distance to the drive portion. Thus, a coupling area between the flex portion and the drive portion, which is possibly thicker as compared to the drive portion, can be moved longitudinally in the area of the funnel geometry/the centering receptacle without it coming into contact with the funnel. This enables a compact design of the tool and the instrument in the longitudinal direction. In other words, the funnel component is positioned/dimensioned such that proximal mounting of the flex portion has sufficient installation space for evasion and the tool is guided into the drive geometry/engagement reception during the coupling process.

In an expedient manner, an axial space is formed in the proximal instrument shaft portion distal to the drive shaft in order to ensure the longitudinal movement of the proximal mounting/coupling area of the flex portion with the drive portion. For this purpose, it is advantageous if a drive train/gear train for bending the instrument shaft is formed radially outside or radially spaced from the tool shaft, e.g. has a ring gear surrounding the axial space.

In particular, the entire drive shaft is located in the proximal instrument shaft portion and optionally in a proximal handle of the surgical instrument. Furthermore, when the tool is fully mounted on the instrument, the entire bearing portion can be arranged in the distal instrument shaft portion and/or the entire drive portion can be arranged in the proximal instrument shaft portion.

Advantageously, the drive shaft forms a longitudinally extending engagement reception, in particular a profile hollow shaft portion, for engagement with the drive portion or its engagement portion. Preferably, the length of the engagement reception corresponds to at least one displacement distance of the proximal end of the flex portion at its maximum curvature. Preferably, the length of the engagement portion of the tool and the length of the engagement reception of the instrument complement each other in such a way that they still overlap even when the flex portion is bent maximally or minimally.

It is also preferred that a distal pin is received in the drive shaft in an axially movable and rotationally fixed manner, which forms a counter profile portion at its distal end for engagement with the profile portion of the surgical tool determined by the rotational position. The counter profile portion is adapted to fit the profile portion in such a way that it can only (fully) engage with the profile portion in one or more specific alignments. For example, the counter profile portion is a negative form of the profile portion or has contact surfaces distributed in the circumferential direction for contact with surfaces of the profile portion. If the rotationally symmetrical tool is coupled in the instrument shaft, it is brought into the correct position (rotational position) via such a pin. Here, it is particularly advantageous to hold the tool on the bearing bush, as the tool shaft can then rotate freely. Due to the interaction/guidance of the profile portion and the counter profile portion, the tool rotates into the correct position (is aligned in the correct position) so that, for example, the engagement portion (e.g. the hexagonal profile) is aligned to match the engagement reception (e.g. an internal hexagonal profile). This simplifies the coupling process and brings the tool (especially in the area of the funnel) into the correct position, so that misalignment of the engagement portion with the engagement reception and tilting in between is largely prevented.

In an expedient manner, a spring element can be provided in the drive shaft proximal to the distal pin, which is adapted to pretension the distal pin in the distal direction. If the tool is not coupled to the instrument, it can thus be ensured that the distal pin or a distal end thereof is held in the area of the funnel or even further distally in order to enable alignment of the engagement portion to the engagement reception at an early stage. The spring element thus provides guidance of the distal pin. In particular, the spring/spring element has a very low spring rate (is a very soft spring) in order to enable easy insertion of the tool and moreover avoid excessive stress on the flex portion. The spring can be a mechanical spring, for example a coil spring, or also a pneumatic spring, for example.

In addition, a proximal pin can preferably be provided proximal to the spring element, which is accommodated (in particular in a rotationally fixed manner) in the drive shaft and supports the spring element proximally. Further preferably, the distal pin, the proximal pin and the spring element form a coupling aid and are connected to each other (e.g. welded/glued) in particular in a rotationally fixed manner. The proximal pin and/or the distal pin can have a tool holder, e.g. an adjustment slot, at its/their respective proximal end. The tool holder(s) is/are accessible at least during assembly of the instrument, for example using a screwdriver. Thus, this/these tool holder(s) is/are provided for adjusting a rotational alignment of the counter profile portion relative to the engagement reception of the drive shaft or to the engagement portion of the tool.

In particular, the surgical instrument is a hand instrument having a proximal gripping element for gripping by a user, from which the instrument shaft extends distally.

Alternatively or additionally, the problem underlying the present disclosure is solved by a system comprising the afore-described surgical tool and the afore-described surgical instrument.

### BRIEF DESCRIPTION OF THE DRAWINGS

The present invention is described below with reference to a preferred embodiment. However, this is of illustrative nature only and is not intended to limit the scope of protection of the present invention.

FIG. 1 shows a surgical tool according to a first embodiment of the present disclosure at the bottom and the surgical tool without a bearing bush mounted thereto at the top.

FIG. 2 shows a detailed view of a flex portion of the surgical tool shown in FIG. 1.

FIG. 3 shows a bearing bush of the surgical tool shown in FIG. 1.

FIG. 4 shows a longitudinal sectional view through the surgical tool illustrated in FIG. 1.

FIG. 5 shows the surgical tool illustrated in FIG. 1, which is mounted in a surgical instrument according to an embodiment of the present disclosure, in a straight aligned state (top) and in an angled state (bottom).

FIG. 6 shows a view of a proximal instrument shaft portion of the instrument shown in FIG. 5.

FIG. 7 shows a coupling aid of the instrument shown in FIG. 5 in a state in which the tool shown in FIG. 1 is not mounted (top) and in a state in which the tool is mounted (bottom).

FIG. 8 illustrates a coupling process between the tool shown in FIG. 1 and the instrument shown in FIG. 5.

FIG. 9 illustrates an adjustment of the coupling aid shown in FIG. 7.

### DETAILED DESCRIPTION

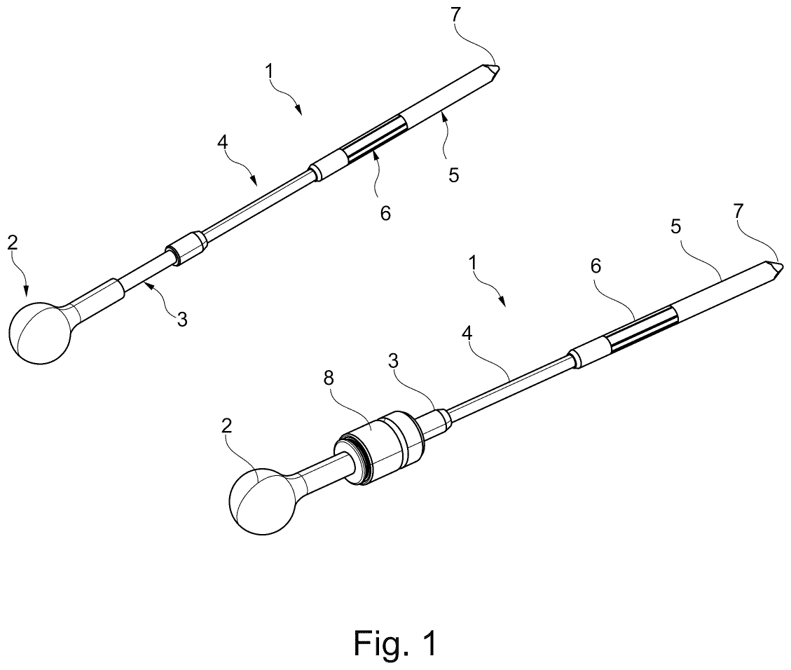

FIG. 1 shows on the left a surgical tool 1 according to a first embodiment of the present disclosure. The tool 1 has a distal tool tip 2, e.g. a cutting head. A tool shaft extends proximally from the tool tip 2. The tool shaft has a bearing portion 3 (directly) connected to the tool tip, which in turn is (directly) connected to a more proximal flex portion 4, which is (directly) connected to a farthest proximal drive portion 5. The drive portion 5 has an engagement portion 6, which, as an example, is formed as a hexagonal profile. A proximal end of the drive portion 5 forms a centering end 7, more precisely, a centering tip with a profile portion in the form of a pyramid extending in the longitudinal direction. The bearing portion 3 carries a bearing bush 8, which is described in more detail below with reference to FIG. 3.

At the top of FIG. 1, the surgical tool is shown without the bearing bush 8. It is discernible that the bearing portion 3 forms a portion with a reduced diameter, which is limited proximally by a radial step of bearing portion 3 and distally by a radial projection of a proximal end of tool tip 2. In the illustration, the bearing bush 8 is fixed to the reduced diameter section on the right.

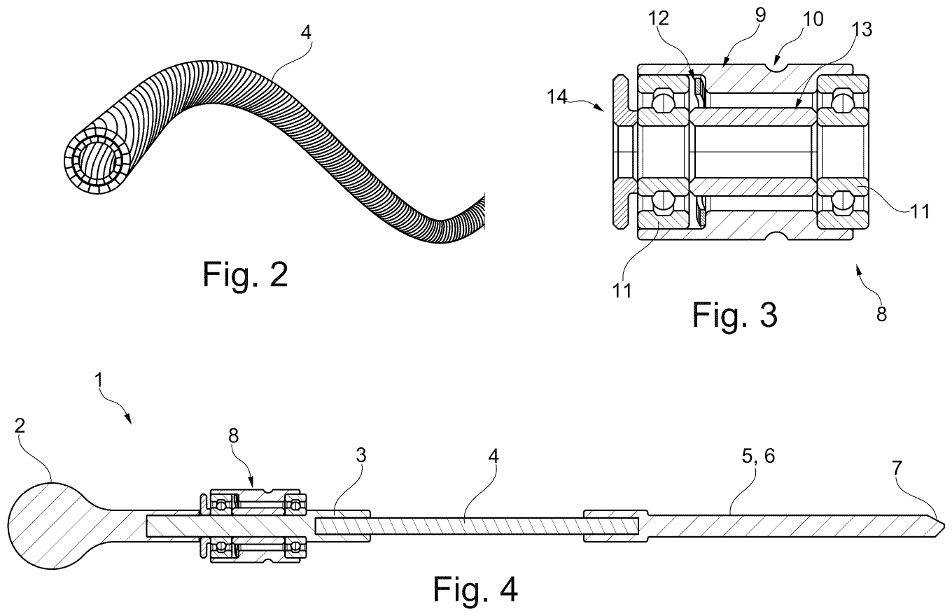

FIG. 2 shows a detailed view of a flex portion 4 of the surgical tool 1 illustrated in FIG. 1. The flex portion 4 has two radial layers. A radially inner layer is formed by fibers/strands that are wound around a longitudinal axis of the tool 1 in a first winding direction. A radially outer layer is formed by fibers/strands that are wound in a second winding direction, opposite to the first winding direction, around the longitudinal axis of the tool 1.

FIG. 3 shows a bearing bush 8 of the surgical tool 1 shown in FIG. 1. The bearing bush 8 has a housing 9 forming a radial outer wall of the bearing bush 8. A circumferential groove is formed in the radial outer wall as an engagement element or as a bearing coupling element 10. A ball groove bearing 11 (in short: bearing) is mounted in the housing 9 at both ends of the housing 9. One of the bearings 11 (the proximally arranged bearing) is fixed to the housing 9 while the other bearing 11 (the distally arranged bearing) is mounted floating therein. A preload element 12 in the form of a wave spring washer is arranged between a housing shoulder of the housing 9 and the floatingly mounted bearing 11, which preloads the corresponding bearing 11, in particular its outer ring, outwards in the axial direction (towards the tool tip in the mounted state). A spacer bushing 13 is provided between the bearings 11, in particular adjacent to their inner rings, which determines the axial position of the bearings 11 relative to one another. Opposite the spacer bushing 13, on an outer side of the bearing bush 8, a cover disk 14 is mounted on the bearing 11 aligned distally on the tool 1, which covers and/or seals a gap between the inner and outer ring of the respective bearing 11.

FIG. 4 is a longitudinal sectional view of the surgical tool 1 shown in FIG. 1, in which it can be seen that the ends of the flex portion 4 each are accommodated in receptions formed at the proximal end of the bearing portion 3 and respectively at the distal end of the drive portion 5. Moreover, it is discernible that the bearing bush 8 is fixed in position in the axial direction by radial shoulders, which are formed respectively by the tool tip 2 and the bearing portion 3.

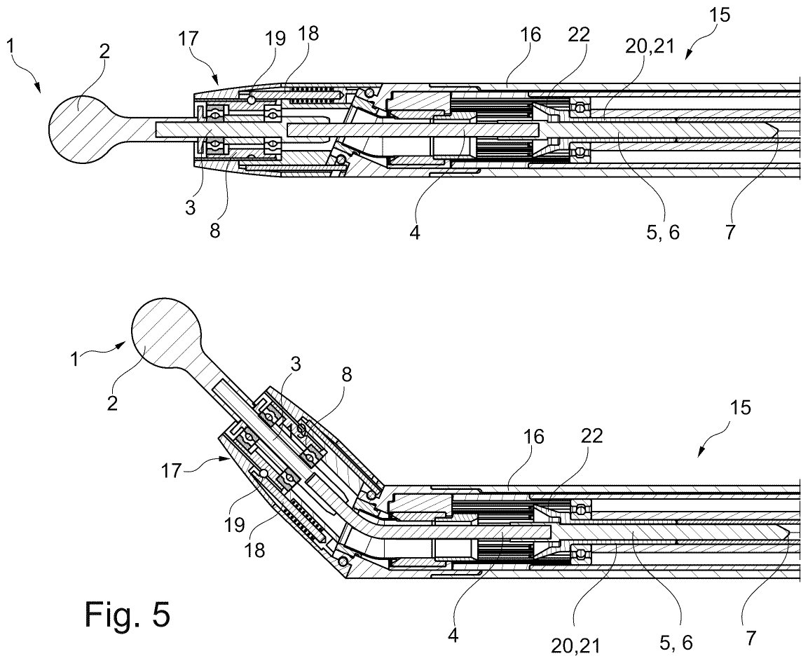

FIG. 5 shows the surgical tool 1 illustrated in FIG. 1, which is mounted in a surgical instrument 15 according to an embodiment of the present disclosure, in a straight/stretched state (top) and in an angled state (bottom). The instrument 15 has a proximal (instrument) shaft portion 16 and a distal (instrument) shaft portion 17, the mutually facing end faces of which extend obliquely with regard to the longitudinal axis and are connected to each other via a rotation mechanism, such that rotation of the proximal and distal shaft portions 16, 17 relative to each other results in an angulation thereof, as shown in the lower illustration.

The tool 1 is fixed in position in the axial direction with respect to the instrument 15 via the bearing bush 8. The bearing bush 8 or the proximal grooved ball bearing 11 forms a stop surface on its proximal end face, with which the bearing bush 8 is in contact with a housing of the distal shaft portion 17. The housing of the distal shaft portion 17 moreover accommodates a latching mechanism including a spring-loaded slider 18, which prestresses a latching body 19 radially inwards for engagement with the engagement element or the bearing coupling element 10 or the groove of the bearing bush 8. The engagement of the latching body 19 in the engagement element or the bearing coupling element 10 also causes to fix a position of the bearing bush 8 relative to the distal shaft portion 17 in the distal direction.

The flex portion 4 extends from bearing portion 3 to drive portion 5 through the instrument shaft 16, 17 and, where necessary (see lower illustration), follows an angle between the proximal and distal shaft portions 16, 17. A proximal end of flex portion 4 is connected to drive portion 5 in the region of an axial space (for example within a ring gear of the rotation mechanism). The drive portion 5, in particular its engagement portion 6, is positively and axially displaceably received in an engagement reception 21 of the drive shaft 20. The engagement reception 21 is complementary to the engagement portion 6, for example in the form of an internal hexagonal profile.

A distal end of the drive shaft 20 forms a funnel-shaped centering reception 22 initially extending radially outwards so as to provide a radial distance between one of the engagement reception 21 and the drive portion 5, so that a radially thickened coupling region at which the flex portion 4 and the drive portion 5 are connected is displaceable within the centering reception 22. Subsequently, the centering reception 22 extends radially widening in the distal direction to form an insertion aid for the drive portion 5 during assembly of the tool 1.

As is apparent when comparing the lower and upper illustrations, an axial position of the tool 1 in distal shaft portion 17 remains constant when the instrument 15 is bent. However, due to the bending of flex portion 4 or slipping thereof in the radial direction during bending, a proximal end of the tool shifts. In particular, the coupling area shifts within the centering reception 22 and the axial space, and the engagement portion 6 of the drive portion 5 shifts in the engagement reception 21 of the drive shaft 22.

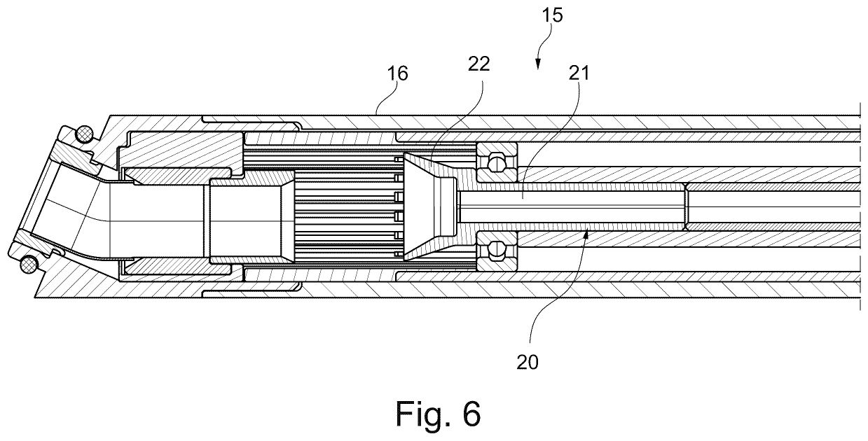

FIG. 6 shows the view of a proximal instrument shaft portion 16 of the instrument shown in FIG. 5, in which the tool 1 is not mounted. Thus, the engagement reception 21 of the drive shaft 22, exemplarily designed here as an internal hexagon, is recognizable.

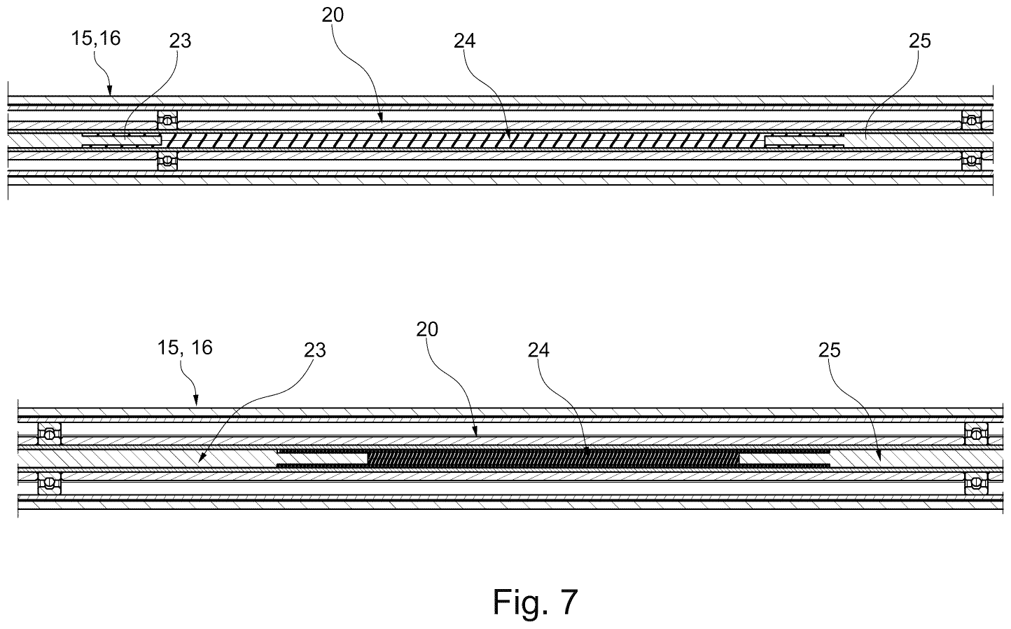

FIG. 7 shows a coupling aid of the instrument shown in FIG. 5 in a state in which the tool shown in FIG. 1 is not mounted (top) and in a state in which the tool is mounted (bottom). The coupling aid has a distal pin 23, a spring element 24 mounted or rotationally fixed at its proximal end and a proximal pin 25 mounted or rotationally fixed again at the proximal end thereof. The distal and proximal pins 23, 25 as well as the spring element 24 are accommodated in the drive shaft 20. The drive shaft 20 may consist of multiple parts. The distal pin 23 is torque-proof and arranged axially displaceable relative to the drive shaft 20. Preferably, also the proximal pin 25 and the spring element 24 are located therein in a torque-proof manner. The spring element 24 pretensions the distal pin 23 in the distal direction. As a result, its distal end protrudes out of the drive shaft 20, as will be explained in more detail below, when the tool 1 is not mounted and the spring element 24 is not compressed, as can be seen in the upper illustration in FIG. 7. When the tool 1 is mounted, the distal pin 23 is pushed back in the proximal direction by the drive portion 5 of the tool 1 and the spring element 24 is compressed, as can be seen in the lower illustration in FIG. 7.

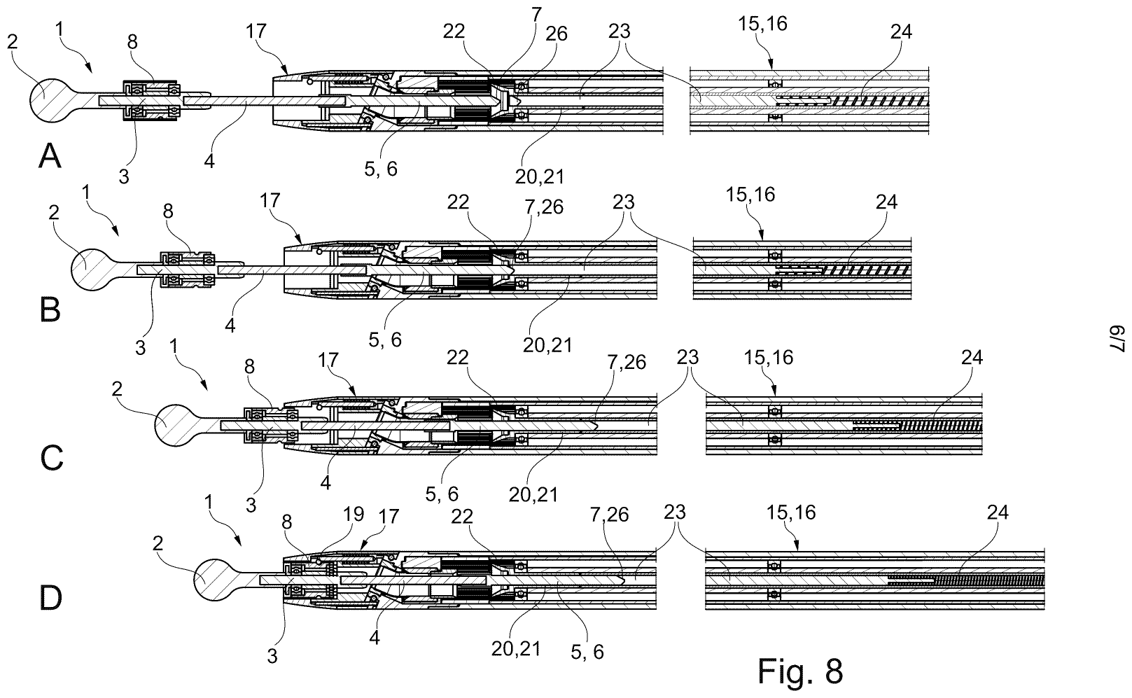

FIG. 8 illustrates a coupling process between the tool shown in FIG. 1 and the instrument shown in FIG. 5. In the top illustration (marked “A”), the tool shaft is introduced into the instrument shaft, but the bearing bush 8 is still outside the instrument shaft. The centering end 7 of the drive portion 7 is introduced into the funnel-shaped centering reception 22 and is centered by it and guided to the engagement reception 21. At the entry of engagement reception 21 the distal end of distal pin 23 is arranged, which forms a counter profile portion 26 for receiving and aligning the profile portion 7 of the drive portion 5. The counter profile portion is aligned with the engagement reception 21 of the drive shaft 20 in such a way that when the profile portion 7 plunges into the counter profile portion 26, as shown in illustration “B”, the engagement portion 6 of the drive portion 5 is aligned with the engagement reception 21 of the drive shaft 20.

Then, as shown in illustration “C” in FIG. 7, the drive portion 5 pushes the distal pin 23 in the proximal direction and the engagement portion 6 of the drive portion of the tool 1 is pushed into the engagement reception 21 of the drive shaft 20 of instrument 15. At the same time, the bearing bush 8 slides into the distal shaft portion 17 of instrument 15. In the lower illustration “D”, the tool 2 is finally fully inserted into the instrument 15. The bearing bush 8 comes into contact with the housing of the distal shaft portion 17 in the proximal direction and the latching body 19 engages in the engagement element or bearing coupling element 10 of the bearing bush 8. The tool 1 is thus fully mounted in the instrument 15.

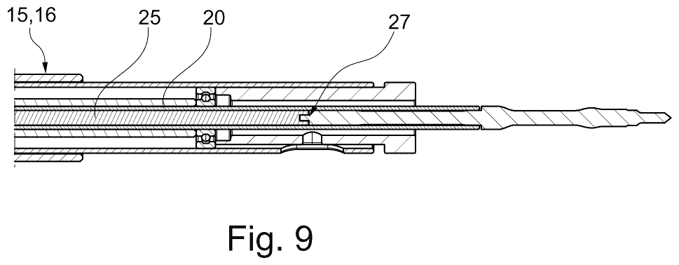

FIG. 9 illustrates an adjustment of the coupling aid shown in FIG. 7. More precisely, a proximal end of proximal pin 25 is shown, on which a herein exemplarily slot-shaped tool holder 27 is provided. During assembly of the instrument, an adjustment tool 28 can be inserted into the tool holder in order to align the proximal pin 25 together with the spring element 24 and distal pin 23, which is connected to it in a rotationally fixed manner, and more precisely its counter profile portion, with the engagement holder 21 of the drive shaft 20. Where appropriate, a tool holder for adjusting the distal pin 23 can also be provided directly on the distal pin 23.

### LIST OF REFERENCE SIGNS

1 Tool
2 Tool tip/-head
3 Bearing portion
4 Flex portion
5 Drive portion
6 Engagement portion
7 Centering end/-tip/profile portion
8 Bearing bush
9 Housing of the bearing bush
10 Bearing coupling element/engagement element/groove
11 Bearing/grooved ball bearing
12 Preload element/wave spring washer
13 Spacer bushing
14 Cover disk
15 Instrument
16 Proximal (instrument) shaft portion
17 Distal (instrument) shaft portion
18 Latching slider (latching mechanism)
19 Latching body/spring-mounted engagement element (latching mechanism)
20 Drive shaft
21 Engagement reception
22 Centering reception
23 Distal pin
24 Spring element
25 Proximal pin
26 Counter profile portion
27 Tool holder

1.-15. (canceled)

16. A surgical tool adapted to be inserted into an instrument shaft of a surgical instrument, comprising:
a distal tool tip;
a tool shaft extending from the distal tool tip in a proximal direction and having a distal bearing portion, the tool shaft also comprising a drive portion arranged proximally to the distal bearing portion, the drive portion adapted for positive engagement with a drive shaft of the surgical instrument, the tool shaft further comprising a flexurally flexible and torsionally rigid flex portion arranged between the distal bearing portion and the drive portion; and
a bearing bush which, in a state not inserted into the surgical instrument, is mounted in an axially fixed manner on the distal bearing portion and has a bearing coupling element for releasable coupling with an instrument shaft of the surgical instrument.

17. The surgical tool according to claim 16, wherein the bearing bush has a proximal end face which forms a contact surface for contact in the instrument shaft.

18. The surgical tool according to claim 16, wherein the drive portion forms at least one engagement portion extending in an axial direction.

19. The surgical tool according to claim 18, wherein a length of the at least one engagement portion corresponds to at least a maximum axial shifting distance of a proximal end of the tool shaft at its maximum curvature.

20. The surgical tool according to claim 16, wherein the drive portion forms a proximal centering end.

21. The surgical tool according to claim 20, wherein the proximal centering end forms a profile portion adapted to engage with a counter-profile portion of the surgical instrument in a manner determined by rotational position.

22. A surgical instrument comprising:
an instrument shaft which is open distally and adapted to receive the surgical tool according to claim 16;
a distal instrument shaft portion, in which a resilient preloaded or pre-loadable latching mechanism is provided, which is adapted for releasable coupling with the bearing coupling element of the surgical tool; and
a proximal instrument shaft portion having a drive shaft configured for positive engagement with the drive portion of the surgical tool.

23. The surgical instrument according to claim 22, wherein the drive shaft forms a distal centering reception.

24. The surgical instrument according to claim 22, wherein an axial space is formed in the proximal instrument shaft portion distal to the drive shaft.

25. The surgical instrument according to claim 22, wherein the drive shaft forms a longitudinally extending engagement reception for engagement with the drive portion.

26. The surgical instrument according to claim 22, wherein a distal pin is received in the drive shaft in an axially movable and rotationally fixed manner, which pin forms a counter profile portion at its distal end for engagement with a profile portion of the surgical tool in a manner determined by rotational position.

27. The surgical instrument according to claim 26, wherein a spring element is provided in the drive shaft proximal to the distal pin, which is adapted to preload the distal pin in a distal direction.

28. The surgical instrument according to claim 27, wherein proximal to the spring element a proximal pin is provided which is received in the drive shaft and connected to the distal pin in a rotationally fixed manner, and which is provided at its proximal end with a tool holder for adjusting a rotational alignment of the counter profile portion with respect to the drive shaft.

29. A system comprising:
the surgical instrument according to claim 22;
a surgical tool adapted to be inserted into an instrument shaft of the surgical instrument;
a distal tool tip;
a tool shaft extending from the distal tool tip in a proximal direction and having a distal bearing portion, the tool shaft further comprising a drive portion arranged proximally to the distal bearing portion, the drive portion adapted for positive engagement with a drive shaft of the surgical instrument; and
a bearing bush which, in a state not inserted into the surgical instrument, is mounted in an axially fixed manner on the distal bearing portion and has a bearing coupling element for releasable coupling with an instrument shaft of the surgical instrument.
